(19) United States
(12) Patent Application Publication
(10) Pub. No.: US 2004/0209287 A1
(43) Pub. Date: Oct. 21, 2004

(54) OCT1p, a protein having homology to the organic and sugar transporter family of proteins, and uses thereof

(75) Inventors: Andrew J. Goodearl, Natick, MA (US); Maria Alexandra Glucksmann, Lexington, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/741,259

(22) Filed: Dec. 19, 2003

Related U.S. Application Data
(63) Continuation of application No. 09/911,667, filed on Jul. 24, 2001, now abandoned.
Application No. 09/911,667 is a continuation of application No. 09/342,959, filed on Jun. 29, 1999, now abandoned.
Application No. 09/342,959 is a continuation-in-part of application No. 09/107,932, filed on Jun. 30, 1998, now abandoned.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

Novel OCTlp polypeptides, proteins, and nucleic acid molecules are disclosed. In addition to isolated, full-length OCT-like proteins, the invention further provides isolated OCTlp fusion proteins, antigenic peptides and anti-OCTlp antibodies. The invention also provides OCTlp nucleic acid molecules, recombinant expression vectors containing a nucleic acid molecule of the invention, host cells into which the expression vectors have been introduced and non-human transgenic animals in which an OCTlp gene has been introduced or disrupted. Diagnostic, screening and therapeutic methods utilizing compositions of the invention are also provided.

### RELATED APPLICATION INFORMATION

This application is a continuation-in-part of application Ser. No. 09/107,932, filed Jun. 30, 1998.

### BACKGROUND OF THE INVENTION

The field of the invention is cellular transporter molecules.

In the course of performing their normal physiological functions, many types of cells, including bacterial cells and those in specialized mammalian tissues such as the liver and kidney, transport a variety of molecules across their cell membranes. For example, cells in the proximal tubule of the kidney transport glucose, amino acids, and uric acid across their membranes, and work to eliminate various drugs and toxic substances from the body. These molecules are transported across the cell membranes by specialized cellular transporters.

Recently, genes encoding several putative transporters have been identified. These molecules include OCT-1 (organic cation transporter; Grundemann et al., Nature 372:549-552, 1994), OCT-2 (Okuda et al., Biochem. Biophys. Res. Comm. 224:500-507, 1996), NLT (novel liver-specific transporter; Simonson et al., J. Cell Sci. 107:1067-1072, 1994), and NKT (novel kidney-specific transporter; Lopez-Nieto et al., J. Biol. Chem. 272:6471-6478, 1997). While the sequences of these transporters are not highly conserved (at the amino acid level, OCT-1 and LT are only 30% and 35% identical to NKT, respectively), they do exhibit similar transmembrane (TM) domain hydropathy profiles.

### SUMMARY OF THE INVENTION

The present invention is based, at least in part, on the discovery of a gene encoding OCTlp (OCT-like protein), a transmembrane protein that is predicted to be member of the superfamily of transporter molecules. The OCTlp cDNA described below (SEQ ID NO:1) has a 1644 nucleotide open reading frame (SEQ ID NO:3 (nucleotides 185-1828 of SEQ ID NO:1)), which encodes a 548 amino acid protein (SEQ ID NO:2). OCTlp is predicted to have twelve (12) transmembrane (TM) domains that extend from about amino acids: 86-108 (SEQ ID NO:6); 122-140 (SEQ ID NO:7); 155-175 (SEQ ID NO:8); 182-198 (SEQ ID NO:9); 209-232 (SEQ ID NO:10); 239-258 (SEQ ID NO:11); 315-337 (SEQ ID NO:12); 376-394 (SEQ ID NO:13); 403-421 (SEQ ID NO:14); 428-447 (SEQ ID NO:15); 458-479 (SEQ ID NO:16); and 487-510 (SEQ ID NO:17). The first domain listed (that extending from amino acid residue 86 to amino acid residue 108) extends from the cytoplasmic side of the membrane toward the extracellular side of the membrane; the second domain listed (that extending from amino acid residue 122 to amino acid residue 140) extends from the extracellular side of the membrane toward the cytoplasmic side of the membrane; and so forth.

OCTlp appears to be the human ortholog of rat SVOP, a synaptic vesicle protein (Janz et al., J. Neurosci. 18:9269-81, 1998). SVOP is present in all areas of rat brain and is expressed at a particularly high level in large pyramidal neurons of the cerebral cortex. SVOP expression is observed in synapses. It also appears to be expressed in the cell bodies of cortical pyramidal neurons.

The OCTlp molecules of the present invention are useful as modulating agents in regulating a variety of cellular processes that depend on transport of biological molecules across a cellular membrane, particularly an intracellular membrane, such as the membrane of a synaptic vesicle or other intracellular vesicle, in neuronal cells or endocrine cells. As described below, OCTlp is highly expressed in cells in the brain. Thus, altering the expression or activity of OCTlp (e.g., with small molecules, antisense molecules, or neutralizing antibodies) can alter the concentration of molecules (such as neurotransmitters) present within the cell or in the extracellular spaces around the cell (i.e., on either side of the plasma membrane). Altering the concentrations of these molecules in patients afflicted with certain conditions, including chronic neurological diseases (e.g., neurodegenerative diseases), central nervous system disorders, behavioral disorders, and eating or sleeping disorders, can provide relief from the symptoms associated therewith.

Central nervous system disorders include, but are not limited to cognitive and neurodegenerative disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders, and neuropsychiatric disorders that include, but are not limited to, schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Further central nervous system-related disorders include, for example, those listed in the American Psychiatric Association's Diagnostic and Statistical manual of Mental Disorders (DSM), the most current version of which is incorporated herein by reference in its entirety.

Accordingly, in one aspect, this invention provides isolated nucleic acid molecules encoding OCT-like proteins or biologically active portions thereof, as well as nucleic acid fragments suitable as primers or hybridization probes for the detection of OCTlp-encoding nucleic acids. These molecules and modulators of OCTlp expression or activity can be used to influence an OCTlp-mediated process. OCTlp-mediated processes include processes that are dependent on and/or responsive to, either directly or indirectly, the level of OCTlp mRNA expression, OCTlp protein expression, or OCTlp activity. Such processes can include, but are not limited to neurotransmission, developmental, cognitive and autonomic neural and neurological processes, such as, for example, pain, appetite, long term memory, and short term memory.

The invention features a nucleic acid molecule that is at least 65% (e.g., 70%, 75%, 80%, 85%, 90%, 95%, or 98%) identical to the nucleotide sequence shown in SEQ ID NO:1, or SEQ ID NO:3, or a complement thereof.

The invention features a nucleic acid molecule that includes a fragment of at least 400 (e.g., 425, 450, 500, 550, 600, 650, 700, 800, 900, 1000, 1200, 1400 or 1600) nucleotides of the nucleotide sequence shown in SEQ ID NO:1 or SEQ ID NO:3, or a complement thereof.

The invention also features a nucleic acid molecule that includes a nucleotide sequence encoding a protein having an amino acid sequence that is at least 75% (e.g., 80%, 85%, 90%, 95%, or 98%) identical to the amino acid sequence of SEQ ID NO:2.

In a preferred embodiment, an OCTlp nucleic acid molecule has the nucleotide sequence shown in SEQ ID NO:1 or SEQ ID NO:3.

Also within the invention is a nucleic acid molecule that encodes a fragment of a polypeptide having the amino acid sequence of SEQ ID NO:2, the fragment including at least 15 (e.g., 20, 25, 30, 50, 100, 150, 300, 400, or 500) contiguous amino acid residues of SEQ ID NO:2.

The invention includes a nucleic acid molecule that encodes a naturally occurring allelic variant of a polypeptide comprising the amino acid sequence of SEQ ID NO:2, wherein the nucleic acid molecule hybridizes to a nucleic acid molecule comprising SEQ ID NO:1 or SEQ ID NO:3 under stringent conditions.

Also within the invention are: an isolated OCT-like protein that is encoded by a nucleic acid molecule having a nucleotide sequence that is at least about 65%, preferably 70%, 80%, 85%, 95%, or 98% identical to SEQ ID NO:3; and an isolated OCT-like protein that is encoded by a nucleic acid molecule having a nucleotide sequence that hybridizes under stringent hybridization conditions to a nucleic acid molecule having the nucleotide sequence of SEQ ID NO:3.

Also within the invention is a polypeptide that is a naturally occurring allelic variant of a polypeptide that includes the amino acid sequence of SEQ ID NO:2, wherein the polypeptide is encoded by a nucleic acid molecule that hybridizes to a nucleic acid molecule comprising SEQ ID NO:1 or SEQ ID NO:3 under stringent conditions.

Another embodiment of the invention features OCTlp nucleic acid molecules that specifically detect OCTlp nucleic acid molecules relative to nucleic acid molecules encoding other members of the superfamily of genes that encodes transporter molecules. For example, in one embodiment, an OCTlp nucleic acid molecule hybridizes under stringent conditions to a nucleic acid molecule comprising the nucleotide sequence of SEQ ID NO:1, SEQ ID NO:3, or a complement thereof. In another embodiment, the OCTlp nucleic acid molecule is at least 300 (e.g., 325, 350, 375, 400, 425, 450, 500, 550, 600, 650, 700, 800, 900, 1000, 1200, 1400, 1600, 2000, 2500, or 3000) nucleotides in length and hybridizes under stringent conditions to a nucleic acid molecule comprising the nucleotide sequence shown in SEQ ID NO:1, SEQ ID NO:3, or a complement thereof. In another embodiment, the invention provides an isolated nucleic acid molecule that is antisense to the coding strand of an OCTlp nucleic acid.

Another aspect of the invention provides a vector, e.g., a recombinant expression vector, comprising an OCTlp nucleic acid molecule of the invention. In another embodiment, the invention provides a host cell containing such a vector. The invention also provides a method for producing an OCT-like protein by culturing, in a suitable medium, a host cell of the invention containing a recombinant expression vector such that an OCT-like protein is produced.

Another aspect of this invention features isolated or recombinant OCT-like proteins and polypeptides. Preferred OCT-like proteins and polypeptides possess at least one biological activity possessed by naturally occurring human OCTlp, e.g., the ability to transport biological molecules (e.g., sugars, neurotransmitters, or small organic molecules) across a cellular membrane. The cellular membrane can be the plasma membrane or the membrane of an intracellular organelle or vesicle, e.g., a synaptic vesicle. Other activities of the polypeptide of the invention may include modulation of cellular proliferation or differentiation, as least in so far as these events depend on transport of metabolites or other factors that are transported into or within the cell by OCTlp.

The OCT-like proteins of the present invention, or biologically active portions thereof, can be operatively linked to a non-OCTlp polypeptide (i.e., heterologous amino acid sequence(s)) to form an OCTlp fusion protein.

The invention further features antibodies that specifically bind OCT-like proteins, such as monoclonal or polyclonal antibodies. In addition, OCT-like proteins or biologically active portions thereof can be incorporated into pharmaceutical compositions, which optionally include pharmaceutically acceptable carriers.

In another aspect, the present invention provides a method for detecting the presence of OCTlp activity or expression in a biological sample by contacting the biological sample with an agent capable of detecting an indicator of OCTlp activity or expression such that the presence of OCTlp activity or expression is detected in the biological sample.

In another aspect, the invention provides a method for modulating OCTlp activity comprising contacting a cell with an agent that modulates (inhibits or stimulates) OCTlp activity or expression such that OCTlp activity or expression in the cell is modulated. In one embodiment, the agent is an antibody that specifically binds to OCT-like protein. In another embodiment, the agent modulates expression of OCTlp by modulating transcription of an OCTlp gene, splicing of an OCTlp mRNA, or translation of a OCTlp mRNA. In yet another embodiment, the agent is a nucleic acid molecule having a nucleotide sequence that is antisense to the coding strand of the OCTlp mRNA or the OCTlp gene.

In one embodiment, the methods of the present invention are used to treat a subject having a disorder characterized by aberrant expression or activity of either OCT-like protein or nucleic acid by administering an agent that is an OCTlp modulator to the subject. In one embodiment, the OCTlp modulator is an OCT-like protein. In another embodiment the OCTlp modulator is an OCTlp nucleic acid molecule. In other embodiments, the OCTlp modulator is a peptide, peptidomimetic, or other small molecule. In a preferred embodiment, the disorder characterized by aberrant OCTlp or nucleic acid expression or related to OCTlp function, e.g., neurotransmission, is a chronic neurological disorder, a neurodegenerative disease (e.g., Alzheimer's Disease, Parkinson's Disease, Huntington's Disease, or amyotrophic lateral sclerosis), a behavioral disorder, an eating disorder, or a sleep disorder.

The present invention also provides a diagnostic assay for identifying the presence or absence of a genetic lesion or mutation characterized by at least one of: (i) aberrant modification or mutation of a gene encoding an OCT-like protein; (ii) mis-regulation of a gene encoding an OCT-like protein; and (iii) aberrant post-translational modification of an OCT-like protein, wherein a wild-type form of the gene encodes a protein with an OCTlp activity.

In another aspect, the invention provides a method for identifying a compound that binds to or modulates the activity of an OCT-like protein. In general, such methods entail measuring a biological activity of an OCT-like protein in the presence and absence of a test compound and identifying those compounds that alter the activity of the OCT-like protein.

The invention also features methods for identifying a compound that modulates the expression of OCTlp by measuring the expression of OCTlp in the presence and absence of a compound.

Other features and advantages of the invention will be apparent from the following detailed description and claims.

### BRIEF DESCRIPTION OF THE DRAWINGS

FIG. 1 depicts the cDNA sequence (SEQ ID NO:1) and predicted amino acid sequence (SEQ ID NO:2) of human OCTlp (also referred to as “OCT-like protein”). The open reading frame of SEQ ID NO:1 extends from nucleotide 185 to nucleotide 1644 (SEQ ID NO:3).

FIG. 2 depicts an alignment of a portion of the amino acid sequence of OCTlp (SEQ ID NO:4, which corresponds to amino acid residues 71 to 524 of SEQ ID NO:2) and a consensus sequence for sugar and other transporters derived from a hidden Markov model (PF00083; SEQ ID NO:5).

FIG. 3 is a hydropathy plot of human OCTlp. The location of the predicted transmembrane (TM), cytoplasmic (IN), and extracellular (OUT) domains are indicated, as are the position of cysteines (cys; vertical bars immediately below the plot). Extensions of the plot above the dotted line represent regions of the protein having a positive hydrophobic index, whereas extensions below the dotted line represent regions of the protein having a negative hydrophobic index (i.e., hydrophilic regions).

FIG. 4 depicts a partial cDNA sequence (SEQ ID NO:20) and predicted amino acid sequence (SEQ ID NO:21) of rat OCTlp.

### DETAILED DESCRIPTION OF THE INVENTION

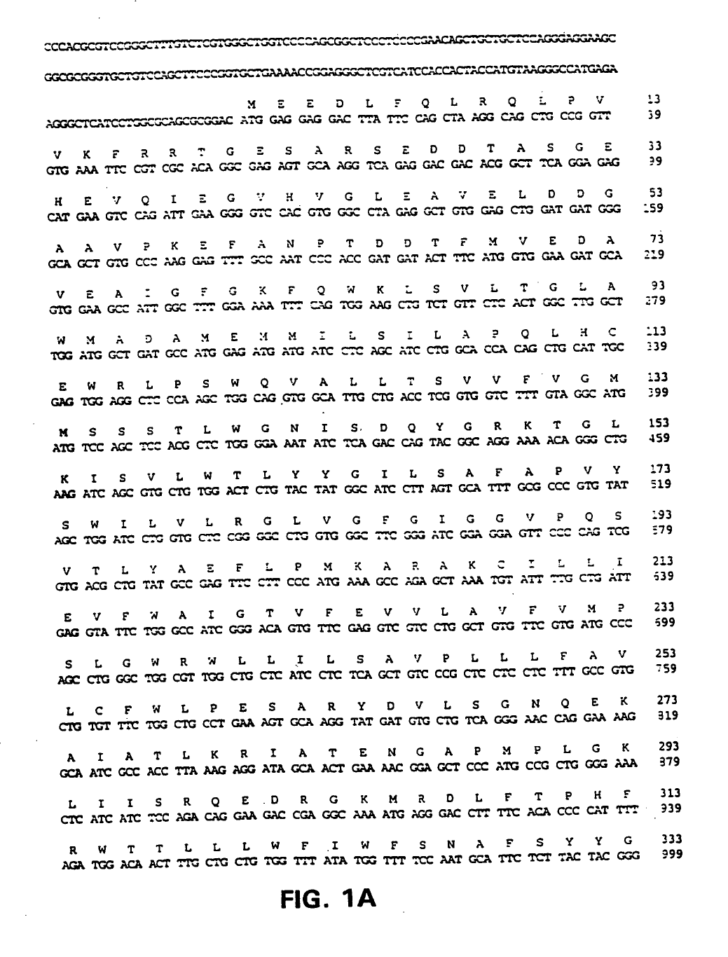

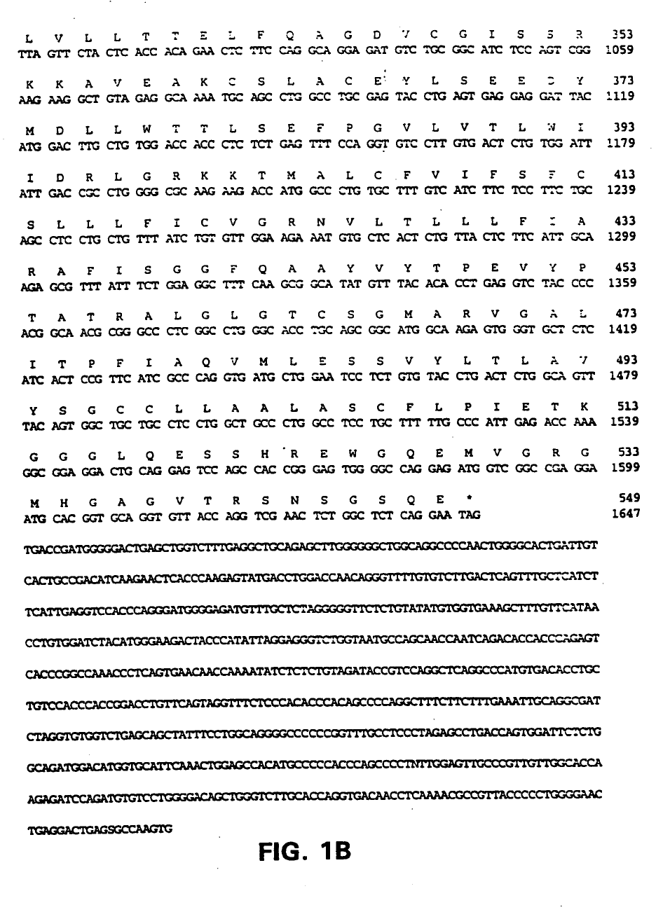

The present invention is based on the discovery of a cDNA molecule encoding human OCTlp, a member of the superfamily of sugar and other transporter molecules that have 12 transmembrane domains. A nucleotide sequence encoding a human OCT-like protein is shown in FIG. 1 (SEQ ID NO:1; SEQ ID NO:3 includes the open reading frame only). A predicted amino acid sequence of OCT-like protein is also shown in FIG. 1 (SEQ ID NO:2).

The OCTlp cDNA of FIG. 1 (SEQ ID NO:1), which is approximately 2562 nucleotides long, including untranslated regions, encodes a protein having a molecular weight of approximately 60 kDa (excluding post-translational modifications).

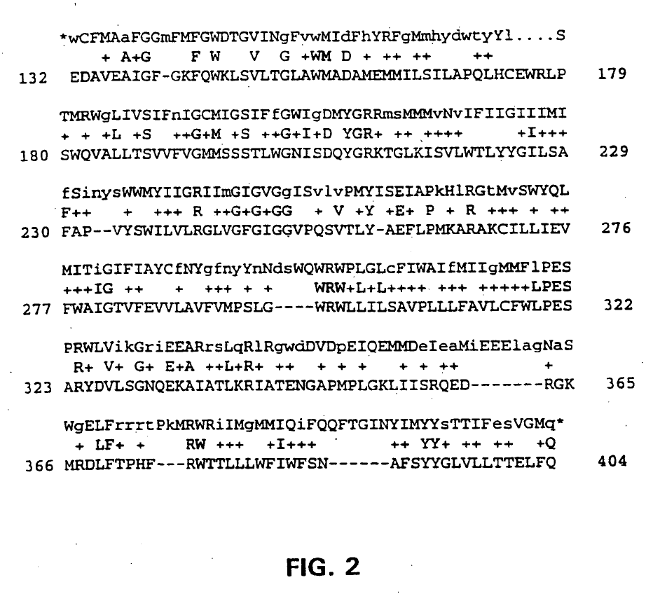

A region of human OCT-like protein (SEQ ID NO:2) bears some similarity to a consensus sequence for sugar and other transporter molecules derived from a hidden Markov model (PF00083; SEQ ID NO:5). This comparison is depicted in FIG. 2.

Northern blot analysis reveals that an approximately 3.0 kb OCTlp mRNA transcript is highly expressed in brain tissue. On the same Northern blot, a lower level of expression of this transcript was observed in testes, while there appeared to be little or no expression in heart, spleen, lung, liver, skeletal muscle, or kidney.

Human OCTlp is one member of a family of molecules (“the OCTlp family”) having certain conserved structural and functional features. The term “family,” when used in reference to the protein and nucleic acid molecules of the invention, is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

As used interchangeably herein, the phrases “OCTlp activity”, “biological activity of OCTlp” or “functional activity of OCTlp”, refer to an activity exerted by an OCT-like protein, polypeptide or nucleic acid molecule on an OCTlp responsive cell as determined in vivo, or in vitro, according to standard techniques. An OCTlp activity can be a direct activity, such as an association with a second protein, or other biological substance, or an indirect activity, such as a cellular signaling activity mediated by interaction of the OCT-like protein with a second protein, or other biological substance.

Various aspects of the invention are described in further detail in the following subsections.

I. Isolated Nucleic Acid Molecules

One aspect of the invention pertains to isolated nucleic acid molecules that encode OCT-like proteins or biologically active portions thereof, as well as nucleic acid molecules sufficient for use as hybridization probes to identify OCTlp-encoding nucleic acids (e.g., OCTlp mRNA) and fragments for use as PCR primers for the amplification or mutation of OCTlp nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one that is separated from other nucleic acid molecules that are present in the natural source of the nucleic acid. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid sequence is derived. For example, in various embodiments, the isolated OCTlp nucleic acid molecule can contain less than about 5 kb (e.g., 4 kb, 3 kb, 2 kb, 1 kb, 0.5 kb or 0.1 kb) of nucleotide sequence that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:3, or a complement of any of these nucleotide sequences, can be isolated using standard molecular biology techniques and the sequence information provided herein. Using all or a portion of the nucleic acid sequences of SEQ ID NO:1, or SEQ ID NO:3 as a hybridization probe, OCTlp nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to OCTlp nucleotide sequences can be prepared by standard synthetic techniques, e.g., using an automated DNA synthesizer.

In another preferred embodiment, an isolated nucleic acid molecule of the invention comprises a nucleic acid molecule that is a complement of the nucleotide sequence shown in SEQ ID NO:1, SEQ ID NO:3, or a portion thereof. A nucleic acid molecule that is complementary to a given nucleotide sequence is one that is sufficiently complementary to the given nucleotide sequence that it can hybridize to the given nucleotide sequence, thereby forming a stable duplex.

Moreover, the nucleic acid molecule of the invention can comprise only a portion of a nucleic acid sequence encoding OCTlp, for example, a fragment that can be used as a probe or primer or a fragment encoding a biologically active portion of OCTlp. The nucleotide sequence determined from the cloning of the human OCTlp gene allows for the generation of probes and primers designed for use in identifying and/or cloning OCTlp homologues in other cell types, e.g., from other tissues, as well as OCTlp homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NO:1, SEQ ID NO:3, or of a naturally occurring mutant of SEQ ID NO:1 or SEQ ID NO:3.

Probes based on the human OCTlp nucleotide sequence can be used to detect transcripts or genomic sequences encoding the same or identical proteins. The probe comprises a label group attached thereto, e.g., a radioisotope, a fluorescent compound, an enzyme, or an enzyme co-factor. Such probes can be used as a part of a diagnostic test kit for identifying cells or tissue that mis-express an OCT-like protein, such as by measuring a level of an OCTlp-encoding nucleic acid in a sample of cells from a subject, e.g., detecting OCTlp mRNA levels or determining whether a genomic OCTlp gene has been mutated or deleted.

A nucleic acid fragment encoding a “biologically active portion of OCTlp” can be prepared by isolating a portion of SEQ ID NO:1 or SEQ ID NO:3, which encodes a polypeptide having an OCTlp biological activity, expressing the encoded portion of OCT-like protein (e.g., by recombinant expression in vitro) and assessing the activity of the encoded portion of OCTlp.

The invention further encompasses nucleic acid molecules that differ from the nucleotide sequence of SEQ ID NO:1 or SEQ ID NO:3 due to degeneracy of the genetic code and thus encode the same OCT-like protein as that encoded by the nucleotide sequence shown in SEQ ID NO:1, or SEQ ID NO:3.

In addition to the human OCTlp nucleotide sequence shown in SEQ ID NO:1 or SEQ ID NO:3, it will be appreciated by those skilled in the art that DNA sequence polymorphisms that lead to changes in the amino acid sequences of OCTlp may exist within a population (e.g., the human population). Such genetic polymorphism in the OCTlp gene may exist among individuals within a population due to natural allelic variation. As used herein, the terms “gene” and “recombinant gene” refer to nucleic acid molecules comprising an open reading frame encoding an OCT-like protein, preferably a mammalian OCT-like protein. Such natural allelic variations can typically result in 1-5% variance in the nucleotide sequence of the OCTlp gene. Any and all such nucleotide variations and resulting amino acid polymorphisms in OCTlp that are the result of natural allelic variation and that do not alter the functional activity of OCTlp are intended to be within the scope of the invention.

Moreover, nucleic acid molecules encoding OCT-like proteins from-other species (OCTlp homologues), which have a nucleotide sequence that differs from that of a human OCTlp, are intended to be within the scope of the invention. Nucleic acid molecules corresponding to natural allelic variants and homologues of the OCTlp cDNA of the invention can be isolated based on their identity to the human OCTlp nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 300 (e.g., 325, 350, 375, 400, 425, 450, 500, 550, 600, 650, 700, 800, 900, 1000, 1200, 1400, 1600, 2000, 2500, or 3000) nucleotides in length and hybridizes under stringent conditions to the nucleic acid molecule comprising the nucleotide sequence, preferably the coding sequence, of SEQ ID NO:1 or SEQ ID NO:3.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1, or SEQ ID NO:3, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

In addition to naturally-occurring allelic variants of the OCTlp sequence that may exist in the population, the skilled artisan will further appreciate that changes can be introduced by mutation into the nucleotide sequence of SEQ ID NO:1, or SEQ ID NO:3, thereby leading to changes in the amino acid sequence of the encoded OCT-like protein, without altering the functional ability of the OCT-like protein. For example, one can make nucleotide substitutions leading to amino acid substitutions at “non-essential” amino acid residues. A “non-essential” amino acid residue is a residue that can be altered from the wild-type sequence of OCTlp (e.g., the sequence of SEQ ID NO:2) without altering the biological activity of the protein, whereas an “essential” amino acid residue is required for biological activity. For example, amino acid residues that are conserved among the OCT-like proteins of various species are predicted to be particularly unamenable to alteration. However, other amino acid residues (e.g., those that are not conserved or only semi-conserved among OCT-like proteins of various species) may not be essential for activity and thus are likely to be amenable to alteration.

Accordingly, another aspect of the invention pertains to nucleic acid molecules encoding OCT-like proteins that contain changes in amino acid residues that are not essential for activity. Such OCT-like proteins differ in amino acid sequence from SEQ ID NO:2 yet retain biological activity. In one embodiment, the isolated nucleic acid molecule includes a nucleotide sequence encoding a protein that includes an amino acid sequence that is at least about 45% identical, 65%, 75%, 85%, 95%, or 98% identical to the amino acid sequence of SEQ ID NO:2.

An isolated nucleic acid molecule encoding an OCT-like protein having a sequence that differs from that of SEQ ID NO:2 can be created by introducing one or more nucleotide substitutions, additions or deletions into the nucleotide sequence of SEQ ID NO:1, or SEQ ID NO:3, such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in OCTlp is preferably replaced with another amino acid residue from the same side chain family. Alternatively, mutations can be introduced randomly along all or part of an OCTlp coding sequence, such as by saturation mutagenesis, and the resultant mutants can be screened for OCTlp biological activity to identify mutants that retain activity. Following mutagenesis, the encoded protein can be expressed recombinantly and the activity of the protein can be determined.

In a preferred embodiment, a mutant OCT-like protein can be assayed for the ability to transport a molecule across a cellular membrane, particularly where the molecule is the same as that transported by wild-type OCTlp. In yet another preferred embodiment, a mutant OCTlp can be assayed for the ability to modulate cellular proliferation or cellular differentiation.

The present invention encompasses antisense nucleic acid molecules, i.e., molecules that are complementary to a sense nucleic acid molecule encoding a protein, e.g., complementary to the coding strand of a double-stranded cDNA molecule or complementary to an mRNA sequence. Accordingly, an antisense nucleic acid molecule can hydrogen bond to a sense nucleic acid molecule. The antisense nucleic acid molecule can be complementary to an entire OCTlp coding strand, or to only a portion thereof, e.g., all or part of the protein coding region (or open reading frame). An antisense nucleic acid molecule can be antisense to a noncoding region of the coding strand of a nucleotide sequence encoding OCTlp. The noncoding regions (“5′ and 3′ untranslated regions”) are the 5′ and 3′ sequences that flank the coding region and are not translated into amino acid residues.

Given the coding strand sequences encoding OCTlp disclosed herein (e.g., SEQ ID NO:1 or SEQ ID NO:3), antisense nucleic acids of the invention can be designed according to the rules of Watson and Crick base pairing. The antisense nucleic acid molecule can be complementary to the entire coding region of OCTlp mRNA, but more preferably is an oligonucleotide that is antisense to only a portion of the coding or noncoding region of OCTlp mRNA. For example, the antisense oligonucleotide can be complementary to the region surrounding the translation start site of OCTlp mRNA, e.g., an oligonucleotide having the sequence 5′-TCCATGTCCGCGCTGCGCCAGGAT-3′ (SEQ ID NO:18) or 5′-TTAGCTGGAATAAGTCCTC-3′ (SEQ ID NO:19). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphoro-thioate derivatives and acridine substituted nucleotides can be used. Examples of modified nucleotides that can be used to generate the antisense nucleic acid include 5-fluoro-uracil, 5-bromouracil, 5-chlorouracil, 5-iodouracil, hypoxanthine, xanthine, 4-acetylcytosine, 5-(carboxy-hydroxylmethyl)uracil, 5-carboxymethyl-aminomethyl-2-thiouridine, 5-carboxymethylaminomethyluracil, dihydro-uracil, beta-D-galactosylqueosine, inosine, N6-isopentenyl-adenine, 1-methylguanine, 1-methylinosine, 2,2-dimethyl-guanine, 2-methyladenine, 2-methylguanine, 3-methylcytosine, 5-methylcytosine, N6-adenine, 7-methyl-guanine, 5-methyl-aminomethyluracil, 5-methoxyaminomethyl-2-thiouracil, beta-D-mannosylqueosine, 5′-methoxycarboxy-methyluracil, 5-meth-oxyuracil, 2-methylthio-N6-isopentenyl-adenine, uracil-5-oxyacetic acid (v), wybutoxosine, pseudouracil, queosine, 2-thiocytosine, 5-methyl-2-thiouracil, 2-thiouracil, 4-thio-uracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diamino-purine. Alternatively, the antisense nucleic acid molecule can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an OCT-like protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave OCTlp mRNA transcripts to thereby inhibit translation of OCTlp mRNA. A ribozyme having specificity for an OCTlp-encoding nucleic acid can be designed based upon the nucleotide sequence of an OCTlp cDNA disclosed herein (e.g., SEQ ID NO:1, SEQ ID NO:3). For example, a derivative of a Tetrahymena L-19 IVS RNA can be constructed in which the nucleotide sequence of the active site is complementary to the nucleotide sequence to be cleaved in an OCTlp-encoding mRNA. See, e.g., Cech et al. U.S. Pat. No. 4,987,071; and Cech et al. U.S. Pat No. 5,116,742. Alternatively, OCTlp mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, OCTlp gene expression can be inhibited by targeting nucleotide sequences complementary to the regulatory region of the OCTlp (e.g., the OCTlp promoter and/or enhancers) to form triple helical structures that prevent transcription of the OCTlp gene in target cells. See, generally, Helene (1991) Anticancer Drug Des. 6(6):569-84; Helene (1992) Ann. N.Y. Acad. Sci. 660:27-36; and Maher (1992) Bioassays 14(12):807-15.

In preferred embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93:14670-675.

PNAs of OCTlp can be used in therapeutic and diagnostic applications. For example, PNAs can be used as antisense or antigene agents for sequence-specific modulation of gene expression by, e.g., inducing transcription or translation arrest or inhibiting replication. PNAs of OCTlp can also be used, e.g., in the analysis of single base pair mutations in a gene by, e.g., PNA directed PCR clamping; as ‘artificial restriction enzymes’ when used in combination with other enzymes, e.g., S1 nucleases (Hyrup (1996) supra; or as probes or primers for DNA sequence and hybridization (Hyrup (1996) supra; Perry-O'Keefe et al. (1996) Proc. Natl. Acad. Sci. USA 93:14670-675).

In another embodiment, PNAs of OCTlp can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of OCTlp can be generated that may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) supra). The synthesis of PNA-DNA chimeras can be performed as described in Hyrup (1996) supra and Finn et al. (1996) Nucleic Acids Research 24(17):3357-63. For example, a DNA chain can be synthesized on a solid support using standard phosphoramidite coupling chemistry and modified nucleoside analogs, e.g., 5′-(4-methoxy-trityl)-amino-5′-deoxy-thymidine phosphoramidite, can be used between the PNA and the 5′ end of DNA (Mag et al. (1989) Nucleic Acid Res. 17:5973-88). PNA monomers are then coupled in a stepwise manner to produce a chimeric molecule with a 5′ PNA segment and a 3′ DNA segment (Finn et al. (1996) Nucleic Acids Research 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated OCT-like Proteins and Anti-OCTlp Antibodies

One aspect of the invention pertains to isolated OCT-like proteins, and biologically active portions thereof, as well as polypeptide fragments suitable for use as immunogens to raise anti-OCTlp antibodies. In one embodiment, native OCT-like proteins can be isolated from cells or tissue sources by an appropriate purification scheme using standard protein purification techniques. In another embodiment, OCT-like proteins are produced by recombinant DNA techniques. Alternative to recombinant expression, an OCT-like protein or polypeptide can be synthesized chemically using standard peptide synthesis techniques.

An “isolated” or “purified” protein or biologically active portion thereof is substantially free of cellular material or other contaminating proteins from the cell or tissue source from which the OCT-like protein is derived, or substantially free from chemical precursors or other chemicals when chemically synthesized. The language “substantially free of cellular material” includes preparations of OCT-like protein in which the protein is separated from cellular components of the cells from which it is isolated or recombinantly produced. Thus, preparations of OCT-like proteins that are substantially free of cellular material include preparations of OCT-like protein having less than about 30%, 20%, 10%, or 5% (by dry weight) of non-OCT-like protein (also referred to herein as a “contaminating protein”). When the OCT-like protein or biologically active portion thereof is recombinantly produced, it is also preferably substantially free of culture medium, i.e., culture medium represents less than about 20%, 10%, or 5% of the volume of the protein preparation. When OCT-like protein is produced by chemical synthesis, it is preferably substantially free of chemical precursors or other chemicals, i.e., it is separated from chemical precursors or other chemicals that are involved in the synthesis of the protein. Accordingly such preparations of OCT-like protein have less than about 30%, 20%, 10%, 5% (by dry weight) of chemical precursors or non-OCTlp chemicals.

Biologically active portions of an OCT-like protein include peptides comprising amino acid sequences sufficiently identical to or derived from the amino acid sequence of the OCT-like protein (e.g., the amino acid sequence shown in SEQ ID NO:2), which include fewer amino acid residues than the full length OCT-like proteins, and exhibit at least one activity of an OCT-like protein. Typically, biologically active portions comprise a domain or motif with at least one activity of the OCT-like protein. A biologically active portion of an OCT-like protein can be a polypeptide that is, for example, 10, 25, 50, 100 or more amino acid residues in length.

Moreover, other biologically active portions, in which other regions of the protein are deleted, can be prepared by recombinant techniques and evaluated for one or more of the functional activities of a native OCT-like protein.

Preferred OCT-like protein has the amino acid sequence shown of SEQ ID NO:2. Other useful OCT-like proteins are substantially identical to SEQ ID NO:2 and retain the functional activity of the protein of SEQ ID NO:2 yet differ in amino acid sequence due to natural allelic variation or mutagenesis. Accordingly, a useful OCT-like protein is a protein that includes an amino acid sequence at least about 75%, preferably 85%, 90%, 95%, 97% or 99% identical to the amino acid sequence of SEQ ID NO:2 and retains at least one of the functional activities of the OCT-like protein of SEQ ID NO:2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul ((1990) Proc. Natl. Acad. Sci. USA 87:2264-2268), modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403-410. BLAST nucleotide searches can be performed with the NBLAST program, score=100, wordlength=12, to obtain nucleotide sequences homologous to OCTlp nucleic acid molecules of the invention. BLAST protein searches can be performed with the XBLAST program, score=50, wordlength=3 to obtain amino acid sequences homologous to OCT-like protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides OCTlp chimeric or fusion proteins. As used herein, an OCTlp “chimeric protein” or “fusion protein” comprises an OCTlp polypeptide operatively linked to a non-OCTlp polypeptide. A “OCTlp polypeptide” refers to a polypeptide having an amino acid sequence corresponding to OCTlp, whereas a “non-OCTlp polypeptide” refers to a polypeptide having an amino acid sequence corresponding to a protein that is not substantially identical to the OCT-like protein, e.g., a protein that is different from the OCT-like protein and that is derived from the same or a different organism. Within an OCTlp fusion protein, the OCTlp polypeptide can correspond to all or a portion of an OCT-like protein, preferably at least one biologically active portion of an OCT-like protein. Within the fusion protein, the term “operatively linked” is intended to indicate that the OCTlp polypeptide and the non-OCTlp polypeptide are fused in-frame to each other. The non-OCTlp polypeptide can be fused to the N-terminus or C-terminus of the OCTlp polypeptide.

One useful fusion protein is a GST-OCTlp fusion protein in which the OCTlp sequences are fused to the C-terminus of the GST sequences. Such fusion proteins can facilitate the purification of recombinant OCTlp.

In yet another embodiment, the fusion protein is an OCTlp-immunoglobulin fusion protein in which all or part of OCTlp is fused to sequences derived from a member of the immunoglobulin protein family. The OCTlp-immunoglobulin fusion proteins of the invention can be used as immunogens to produce anti-OCTlp antibodies in a subject, to identify molecules transported by OCTlp, and in screening assays to identify molecules that inhibit or facilitate molecular transport mediated by OCTlp.

Preferably, an OCTlp chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques, including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Current Protocols in Molecular Biology, Ausubel et al. eds., John Wiley & Sons, 1992). Moreover, many expression vectors are commercially available that already encode a fusion moiety (e.g., a GST polypeptide). An OCTlp-encoding nucleic acid can be cloned into such an expression vector such that the fusion moiety is linked in-frame to the OCT-like protein.

The present invention also pertains to variants of the OCT-like proteins that function as either OCTlp agonists (mimetics) or as OCTlp antagonists. Variants of the OCT-like protein can be generated by mutagenesis, e.g., discrete point mutation or truncation of the OCT-like protein. An agonist of the OCT-like protein can retain substantially the same, or a subset, of the biological activities of the naturally occurring form of the OCT-like protein. An antagonist of the OCT-like protein can inhibit one or more of the activities of the naturally occurring form of the OCT-like protein by, for example, competitively binding to a downstream or upstream member of a cellular signaling cascade that is involved in the OCTlp activity. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the OCT-like proteins.

Variants of the OCT-like protein that function as either OCTlp agonists (mimetics) or as OCTlp antagonists can be identified by screening combinatorial libraries of mutants, e.g., truncation mutants, of the OCT-like protein for OCT-like protein agonist or antagonist activity. In one embodiment, a variegated library of OCTlp variants is generated by combinatorial mutagenesis at the nucleic acid level and is encoded by a variegated gene library. A variegated library of OCTlp variants can be produced by, for example, enzymatically ligating a mixture of synthetic oligonucleotides into gene sequences such that a degenerate set of potential OCTlp sequences is expressible as individual polypeptides, or alternatively, as a set of larger fusion proteins (e.g., for phage display) containing the set of OCTlp sequences therein. There are a variety of methods that can be used to produce libraries of potential OCTlp variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential OCTlp sequences. Methods for synthesizing degenerate oligonucleotides are known in the art (see, e.g., Narang (1983) Tetrahedron 39:3; Itakura et al. (1984) Ann. Rev. Biochem. 53:323; Itakura et al. (1984) Science 198:1056; Ike et al. (1983) Nucleic Acid Res. 11:477).

In addition, libraries of fragments of the OCT-like protein coding sequence can be used to generate a variegated population of OCTlp fragments for screening and subsequent selection of variants of an OCT-like protein. In one embodiment, a library of coding sequence fragments can be generated by treating a double stranded PCR fragment of an OCTlp coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived that encodes N-terminal and internal fragments of various sizes of the OCT-like protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of OCT-like proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify OCTlp variants (Arkin and Yourvan (1992) Proc. Natl. Acad. Sci. USA 89:7811-7815; Delgrave et al. (1993) Protein Engineering 6(3):327-331).

An isolated OCT-like protein, or a portion or fragment thereof, can be used as an immunogen to generate antibodies that bind OCTlp using standard techniques for polyclonal and monoclonal antibody preparation. The full-length OCT-like protein can be used or, alternatively, the invention provides antigenic peptide fragments of OCTlp for use as immunogens. The antigenic peptide of OCTlp comprises at least 8 (preferably 10, 15, 20, or 30) amino acid residues of the amino acid sequence shown in SEQ ID NO:2 and encompasses an epitope of OCTlp such that an antibody raised against the peptide forms a specific immune complex with OCTlp.

Preferred epitopes encompassed by the antigenic peptide are regions of OCTlp that are located on the surface of the protein, e.g., hydrophilic regions. A hydrophobicity analysis of the human OCT-like protein sequence indicates that the regions between, e.g., the following amino acid residues are particularly hydrophilic and, therefore, likely to be useful for targeting antibody production: amino acid residues 109-121; 176-181; 233-238; 338-375; 422-427; and 480-486.

An OCTlp immunogen typically is used to prepare antibodies by immunizing a suitable subject (e.g., a rabbit, goat, mouse, or other mammal) with the immunogen. An appropriate immunogenic preparation can contain, for example, recombinantly expressed OCT-like protein or a chemically synthesized OCTlp polypeptide. The preparation can further include an adjuvant, such as Freund's complete or incomplete adjuvant, or a similar immunostimulatory agent. Immunization of a suitable subject with an immunogenic OCTlp preparation induces a polyclonal anti-OCTlp antibody response.

Accordingly, another aspect of the invention pertains to anti-OCTlp antibodies. The term “antibody” as used herein refers to immunoglobulin molecules and immunologically active portions of immunoglobulin molecules, i.e., molecules that contain an antigen binding site that specifically binds an antigen, such as OCTlp. A molecule that specifically binds to OCTlp is a molecule that binds OCTlp, but does not substantially bind other molecules in a sample, e.g., a biological sample, which naturally contains OCTlp. Examples of immunologically active portions of immunoglobulin molecules include F(ab) and F(ab′)₂ fragments, which can be generated by treating the antibody with an enzyme such as pepsin. The invention provides polyclonal and monoclonal antibodies that bind OCTlp. The term “monoclonal antibody” or “monoclonal antibody composition”, as used herein, refers to a population of antibody molecules that contain only one species of an antigen binding site capable of immunoreacting with a particular epitope of OCTlp. A monoclonal antibody composition thus typically displays a single binding affinity for a particular OCT-like protein with which it immunoreacts.

Polyclonal anti-OCTlp antibodies can be prepared as described above by immunizing a suitable subject with an OCTlp immunogen. The anti-OCTlp antibody titer in the immunized subject can be monitored over time by standard techniques, such as with an enzyme linked immunosorbent assay (ELISA) using immobilized OCTlp. If desired, the antibody molecules directed against OCTlp can be isolated from the mammal (e.g., from the blood) and further purified by well-known techniques, such as protein A chromatography to obtain the IgG fraction. At an appropriate time after immunization, e.g., when the anti-OCTlp antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies from monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an OCTlp immunogen as described above, and the culture supernatants of the resulting hybridoma cells are screened to identify a hybridoma producing a monoclonal antibody that binds OCTlp.

Any of the many well known protocols used for fusing lymphocytes and immortalized cell lines can be applied for the purpose of generating an anti-OCTlp monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Agl4 myeloma lines. These myeloma lines are available from the ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind OCTlp, e.g., using a standard ELISA assay.

Alternative to preparing monoclonal antibody-secreting hybridomas, a monoclonal anti-OCTlp antibody can be identified and isolated by screening a recombinant combinatorial immunoglobulin library (e.g., an antibody phage display library) with OCTlp to thereby isolate immunoglobulin library members that bind OCTlp. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734.

Additionally, recombinant anti-OCTlp antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-OCTlp antibody (e.g., monoclonal antibody) can be used to isolate OCTlp by standard techniques, such as affinity chromatography or immunoprecipitation. An anti-OCTlp antibody can facilitate the purification of natural OCTlp from cells as well as the purification of recombinantly produced OCTlp expressed in host cells. Moreover, an anti-OCTlp antibody can be used to detect OCT-like protein (e.g., in a cellular lysate or cell supernatant) in order to evaluate the abundance and pattern of expression of the OCT-like protein. Anti-OCTlp antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid molecule encoding OCTlp (or a portion thereof). As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., OCT-like proteins, mutant forms of OCTlp, fusion proteins, etc.).

The recombinant expression vectors of the invention can be designed for expression of OCTlp in prokaryotic or eukaryotic cells, e.g., bacterial cells such as E. coli, insect cells (using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid molecule to be inserted into an expression vector so that the individual codons for each amino acid residue are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the OCTlp expression vector is a yeast expression vector. Examples of vectors for expression in yeast S. cerivisae include pYepSecl (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and picz (InVitrogen Corp, San Diego, Calif.).

Alternatively, OCTlp can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells see chapters 16 and 17 of Sambrook et al. (supra).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to OCTlp mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, OCT-like protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid molecule encoding a selectable marker can be introduced into a host cell on the same vector as that encoding OCTlp or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) OCT-like protein. Accordingly, the invention further provides methods for producing OCT-like protein using the host cells of the invention. In one embodiment, the method comprises culturing a host cell of the invention (into which a recombinant expression vector encoding OCTlp has been introduced) in a suitable medium such that OCT-like protein is produced. In another embodiment, the method further comprises isolating OCTlp from the medium or the host cell.

The host cells of the invention can also be used to produce nonhuman transgenic animals. For example, in one embodiment, a host cell of the invention is a fertilized oocyte or an embryonic stem cell into which OCTlp-coding sequences have been introduced. Such host cells can then be used to create non-human transgenic animals in which exogenous OCTlp sequences have been introduced into their genome or homologous recombinant animals in which endogenous OCTlp sequences have been altered. Such animals are useful for studying the function and/or activity of OCTlp and for identifying and/or evaluating modulators of OCTlp activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA, which is integrated into the genome of a cell from which a transgenic animal develops and that remains in the genome of the mature animal, thereby directing the expression of an encoded gene product in one or more cell types or tissues of the transgenic animal. As used herein, an animal referred to as a “homologous recombinant animal” is a non-human animal, preferably a mammal, more preferably a mouse, in which an endogenous OCTlp gene has been altered by homologous recombination between the endogenous gene and an exogenous DNA molecule introduced into a cell of the animal, e.g., an embryonic cell of the animal, prior to development of the animal.

A transgenic animal of the invention can be created by introducing OCTlp-encoding nucleic acid into the male pronuclei of a fertilized oocyte, e.g., by microinjection, retroviral infection, and allowing the oocyte to develop in a pseudopregnant female foster animal. The OCTlp cDNA sequence e.g., that of (SEQ ID NO:1 or SEQ ID NO:3) can be introduced as a transgene into the genome of a non-human animal. Alternatively, a nonhuman homologue of the human OCTlp gene, such as a mouse OCTlp gene, can be isolated based on hybridization to the human OCTlp cDNA and used as a transgene. Intronic sequences and polyadenylation signals can also be included in the transgene to increase the efficiency of expression of the transgene. A tissue-specific regulatory sequence(s) can be operably linked to the OCTlp transgene to direct expression of OCT-like protein to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan, Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the OCTlp transgene in its genome and/or expression of OCTlp mRNA in tissues or cells of the animals.

A transgenic founder animal can then be used to breed additional animals carrying the transgene. Moreover, transgenic animals carrying a transgene encoding OCTlp can further be bred to other transgenic animals carrying other transgenes.

To create a homologous recombinant animal, a vector is prepared that contains at least a portion of an OCTlp gene (e.g., a human or a non-human homolog of the OCTlp gene, e.g., a murine OCTlp gene) into which a deletion, addition or substitution has been introduced to thereby alter, e.g., functionally disrupt, the OCTlp gene. In a preferred embodiment, the vector is designed such that, upon homologous recombination, the endogenous OCTlp gene is functionally disrupted (i.e., no longer encodes a functional protein; also referred to as a “knock out” vector). Alternatively, the vector can be designed such that, upon homologous recombination, the endogenous OCTlp gene is mutated or otherwise altered but still encodes functional protein (e.g., the upstream regulatory region can be altered to thereby alter the expression of the endogenous OCT-like protein). In the homologous recombination vector, the altered portion of the OCTlp gene is flanked at its 5′ and 3′ ends by-additional nucleic acid of the OCTlp gene to allow for homologous recombination to occur between the exogenous OCTlp gene carried by the vector and an endogenous OCTlp gene in an embryonic stem cell. The additional flanking OCTlp nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced OCTlp gene has homologously recombined with the endogenous OCTlp gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, see, e.g., Lakso etal. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to the morula stage or into a blastocyte and is then transferred to a pseudo-pregnant female foster animal. The offspring borne of this female foster animal will be a clone of the animal from which the cell, e.g., the somatic cell, is isolated.

IV. Pharmaceutical Compositions

The OCTlp nucleic acid molecules, OCT-like proteins, and anti-OCTlp antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral (e.g., intravenous, intradermal, or subcutaneous administration), oral, transmucosal (e.g., inhalation), transdermal (e.g., topical), and rectal administration. Solutions or suspensions used for parenteral or oral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity, such as sodium chloride or dextrose. The pH of the formulation can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF; Parsippany, N.J.) or phosphate buffered saline (PBS). In all cases, the composition injected must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., an OCT-like protein or anti-OCTlp antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is embedded. Alternatively, where the complete gene delivery vector can be produced intact from recombinant cells, e.g. retroviral vectors, the pharmaceutical preparation can include one or more cells that produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials, and pharmacogenomics); and d) methods of treatment (e.g., therapeutic and prophylactic). An OCT-like protein interacts with other cellular proteins and can thus be used for (i) regulation of cellular proliferation; (ii) regulation of cellular differentiation; and (iii) regulation of cell survival. The isolated nucleic acid molecules of the invention can be used to express OCT-like protein (e.g., via a recombinant expression vector in a host cell in gene therapy applications), to detect OCTlp mRNA (e.g., in a biological sample) or a genetic lesion in an OCTlp gene, and to modulate OCTlp activity. In addition, the OCT-like proteins can be used to screen drugs or compounds that modulate the OCTlp activity or expression as well as to treat disorders related to OCT1p function, e.g., neurotransmission, or characterized by insufficient or excessive production of OCT-like protein or production of OCT-like protein forms that have decreased or aberrant activity compared to OCTlp wild type protein. In addition, the anti-OCTlp antibodies of the invention can be used to detect and isolate OCT-like proteins and modulate OCTlp activity.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) that bind to OCT-like proteins or have a stimulatory or inhibitory effect on, for example, OCTlp expression or OCTlp activity.

In one embodiment, the invention provides assays for screening candidate or test compounds that bind to or modulate the activity of the membrane-bound form of an OCT-like protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is a cell-based assay in which a cell that expresses a membrane-bound form of OCT-like protein, or a biologically active portion thereof, on the cell surface is contacted with a test compound and the ability of the test compound to bind to the OCT-like protein is determined. The cell, for example, can be a cell of mammalian origin, a mammalian oocyte such as from Xenopus, or single cell eukaryotic systems such as yeast. Determining the ability of the test compound to bind to the OCT-like protein can be accomplished, for example, by coupling the test compound to a label (e.g., a radioisotope or enzymatic label) such that binding of the test compound to the OCT-like protein, or biologically active portion thereof, can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell that expresses a membrane-bound form of OCT-like protein, or a biologically active portion thereof, with a test compound and a biological substance that OCTlp is capable of transporting across the cell membrane, and determining the ability of the test compound to interact with the OCT-like protein by determining the ability of the test compound to alter the ability of OCTlp to transport the biological substance across the membrane. That is, one can determine the ability of the test compound to modulate (e.g., stimulate or inhibit) the activity of the OCT-like protein or biologically active portion thereof. Determining the ability of the test compound to modulate the activity of OCTlp or a biologically active portion thereof can be accomplished, for example, by determining the ability of the OCT-like protein to bind to or interact with an OCTlp target molecule. As used herein, a “target molecule” is a molecule with which an OCT-like protein interacts in nature, for example, a biological substance that enters or exits a membrane-bound entity (such as a cell, organelle, or vesicle) by way of an OCT-like protein. In one embodiment, an OCTlp target molecule is a component of a signalling pathway that facilitates transduction of an extracellular signal (e.g., a signal generated by a neurotransmitter) through the cell membrane and into the cell.

Determining the ability of the OCT-like protein to interact with an OCTlp target molecule can be accomplished by the method described above for determining direct binding. In a preferred embodiment, determining the ability of the OCT-like protein to interact with an OCTlp target molecule can be accomplished by determining the activity of the target molecule. For example, the activity of the target molecule can be determined by detecting induction of a cellular second messenger of the target (e.g., intracellular Ca²⁺, diacylglycerol, inositol trisphosphate, etc.), detecting activity of the target on an appropriate substrate, or detecting a cellular response, for example, cell survival, cellular differentiation, or cell proliferation.

In yet another embodiment, an assay of the present invention can be performed as described above, but in the context of a cell-free membrane-based system, many of which are known in the art.

In another embodiment, modulators of OCTlp expression are identified in a method in which a cell is contacted with a candidate compound and the expression of OCTlp mRNA or protein in the cell is determined. The level of expression of OCTlp mRNA or protein in the presence of the candidate compound is compared to the level of expression of OCTlp mRNA or protein in the absence of the candidate compound. The candidate compound can then be identified as a modulator of OCTlp expression based on this comparison. For example, when expression of OCTlp mRNA or protein is greater (statistically significantly greater) in the presence of the candidate compound than in its absence, the candidate compound is identified as a stimulator of OCTlp mRNA or protein expression. Alternatively, when expression of OCTlp mRNA or protein is less (statistically significantly less) in the presence of the candidate compound than in its absence, the candidate compound is identified as an inhibitor of OCTlp mRNA or protein expression. The level of OCTlp mRNA or protein expression in the cells can be determined by methods described herein for detecting OCTlp mRNA or protein.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, OCTlp nucleic acid molecules described herein or fragments thereof, can be used to map the location of OCTlp genes on a chromosome. The mapping of the OCTlp sequences to chromosomes is an important first step in correlating these sequences with genes associated with disease.

Briefly, OCTlp genes can be mapped to chromosomes by preparing PCR primers (preferably 15-25 bp in length) from the OCTlp sequences. Computer analysis of OCTlp sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the OCTlp sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the OCTlp sequences to design oligonucleotide primers, sublocalization can be achieved with panels of fragments from specific chromosomes. Other mapping strategies that can similarly be used to map an OCTlp sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa.

A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick,Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature, 325:783-787.

Moreover, differences in the DNA sequences between individuals affected and unaffected with a disease associated with the OCTlp gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The OCTlp sequences of the present invention can also be used to identify individuals from minute biological samples. The U.S. military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags”, which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique that determines the actual base-by-base DNA sequence of selected portions of an individual's genome. Thus, the OCTlp sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The OCTlp sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can comfortably provide positive individual identification with a panel of perhaps 10 to 1,000 primers, which each yield a noncoding amplified sequence of 100 bases. If predicted coding sequences, such as those in SEQ ID NO:3 are used, a more appropriate number of primers for positive individual identification would be 500-2,000.

If a panel of reagents from OCTlp sequences described herein is used to generate a unique identification database for an individual, those same reagents can later be used to identify tissue from that individual. Using the unique identification database, positive identification of the individual, living or dead, can be made from extremely small tissue samples.

3. Use of Partial OCTlp Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the OCTlp sequences or portions thereof, e.g., fragments derived from the noncoding regions of SEQ ID NO:1 having a length of at least 20 or 30 bases.

The OCTlp sequences described herein can further be used to provide polynucleotide reagents, e.g., labeled or labelable probes that can be used in, for example, an in situ hybridization technique, to identify a specific tissue, e.g., brain tissue. This can be very useful in cases where a forensic pathologist is presented with a tissue of unknown origin. Panels of such OCTlp probes can be used to identify tissue by species and/or by organ type.

In a similar fashion, these reagents, e.g., OCTlp primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining OCT-like protein and/or nucleic acid expression as well as OCTlp activity, in the context of a biological sample (e.g., blood, serum, cells, tissue) to thereby determine whether an individual is afflicted with a disease or disorder, or is at risk of developing a disorder, associated with aberrant OCTlp expression or activity. The invention also provides for prognostic (or predictive) assays for determining whether an individual is at risk of developing a disorder associated with OCT-like protein, nucleic acid expression or activity. For example, mutations in an OCTlp gene can be assayed in a biological sample. Such assays can be used for prognostic or predictive purpose to thereby prophylactically treat an individual prior to the onset of a disorder characterized by or associated with OCT-like protein, nucleic acid expression or activity.

Another aspect of the invention provides methods for determining OCT-like protein, nucleic acid expression or OCTlp activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of OCTlp in clinical trials.

These and other agents are described in further detail in the following sections.

1. Diagnostic Assays

An exemplary method for detecting the presence or absence of OCTlp in a biological sample involves obtaining a biological sample from a test subject and contacting the biological sample with a compound or an agent capable of detecting OCT-like protein or nucleic acid (e.g., mRNA, genomic DNA) that encodes OCT-like protein such that the presence of OCTlp is detected in the biological sample. A preferred agent for detecting OCTlp mRNA or genomic DNA is a labeled nucleic acid probe capable of hybridizing to OCTlp mRNA or genomic DNA. The nucleic acid probe can be, for example, a full-length OCTlp nucleic acid, such as the nucleic acid of SEQ ID NO:1 or SEQ ID NO:3, or a portion thereof, such as an oligonucleotide of at least 15, 30, 50, 100, 250 or 500 nucleotides in length and sufficient to specifically hybridize under stringent conditions to OCTlp mRNA or genomic DNA. Other suitable probes for use in the diagnostic assays of the invention are described herein.

A preferred agent for detecting OCT-like protein is an antibody capable of binding to OCT-like protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect OCTlp mRNA, protein, or genomic DNA in a biological sample in vitro as well as in vivo. For example, in vitro techniques for detection of OCTlp mRNA include Northern hybridizations and in situ hybridizations. In vitro techniques for detection of OCT-like protein include enzyme linked immunosorbent assays (ELISAs), Western blots, immunoprecipitations and immunofluorescence. In vitro techniques for detection of OCTlp genomic DNA include Southern hybridizations. Furthermore, in vivo techniques for detection of OCT-like protein include introducing into a subject a labeled anti-OCTlp antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting OCT-like protein, mRNA, or genomic DNA, such that the presence of OCT-like protein, mRNA or genomic DNA is detected in the biological sample, and comparing the presence of OCT-like protein, mRNA or genomic DNA in the control sample with the presence of OCT-like protein, mRNA or genomic DNA in the test sample.

The invention also encompasses kits for detecting the presence of OCTlp in a biological sample (a test sample). Such kits can be used to determine if a subject is suffering from or is at increased risk of developing a disorder associated with aberrant expression of OCTlp (e.g., an immunological disorder). For example, the kit can comprise a labeled compound or agent capable of detecting OCT-like protein or mRNA in a biological sample and means for determining the amount of OCTlp in the sample (e.g., an anti-OCTlp antibody or an oligonucleotide probe that binds to DNA encoding OCTlp, e.g., SEQ ID NO:1 or SEQ ID NO:3). Kits may also include instruction for observing that the tested subject is suffering from or is at risk of developing a disorder associated with aberrant expression of OCTlp if the amount of OCT-like protein or mRNA is above or below a normal level.

For antibody-based kits, the kit may comprise, for example: (1) a first antibody (e.g., attached to a solid support) that binds to OCT-like protein; and, optionally, (2) a second, different antibody that binds to OCT-like protein or the first antibody and is conjugated to a detectable agent.

For oligonucleotide-based kits, the kit may comprise, for example: (1) a oligonucleotide, e.g., a detectably labelled oligonucleotide, which hybridizes to an OCTlp nucleic acid sequence or (2) a pair of primers useful for amplifying an OCTlp nucleic acid molecule.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples that can be assayed and compared to the test sample contained. Each component of the kit is usually enclosed within an individual container and all of the various containers are within a single package along with instructions for observing whether the tested subject is suffering from or is at risk of developing a disorder associated with aberrant expression of OCTlp.

2. Prognostic Assays

The methods described herein can furthermore be utilized as diagnostic or prognostic assays to identify subjects having or at risk of developing a disease or disorder associated with aberrant OCTlp expression or activity. For example, the assays described herein, such as the preceding diagnostic assays or the following assays, can be utilized to identify a subject having or at risk of developing a disorder associated with aberrant activity or expression of OCT-like proteins or the nucleic acid molecules encoding them, e.g., a chronic neurological disorder, a neurodegenerative disease (e.g., Alzheimer's Disease, Parkinson's Disease, Huntington's Disease, or amyotrophic lateral sclerosis), a behavioral disorder, an eating disorder, or a sleep disorder. Thus, the present invention provides a method in which a test sample is obtained from a subject and OCT-like protein or nucleic acid (e.g., mRNA, genomic DNA) is detected, wherein the presence of OCT-like protein or nucleic acid is diagnostic for a subject having or at risk of developing a disease or disorder associated with aberrant OCTlp expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant OCTlp expression or activity. For example, such methods can be used to determine whether a subject can be effectively treated with a specific agent or class of agents (e.g., agents of a type that decrease OCTlp activity). Thus, the present invention provides methods for determining whether a subject can be effectively treated with an agent for a disorder associated with aberrant OCTlp expression or activity in which a test sample is obtained and OCT-like protein or nucleic acid is detected (e.g., wherein the presence of OCT-like protein or nucleic acid is diagnostic for a subject that can be administered the agent to treat a disorder associated with aberrant OCTlp expression or activity).

The methods of the invention can also be used to detect genetic lesions or mutations in an OCTlp gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion characterized by at least one of an alteration affecting the integrity of a gene encoding an OCTlp-protein, or the mis-expression of the OCTlp gene. For example, such genetic lesions can be detected by ascertaining the existence of at least one of 1) a deletion of one or more nucleotides from an OCTlp gene; 2) an addition of one or more nucleotides to an OCTlp gene; 3) a substitution of one or more nucleotides of an OCTlp gene, 4) a chromosomal rearrangement of an OCTlp gene; 5) an alteration in the level of a messenger RNA transcript of an OCTlp gene, 6) aberrant modification of an OCTlp gene, such as of the methylation pattern of the genomic DNA, 7) the presence of a non-wild type splicing pattern of a messenger RNA transcript of an OCTlp gene, 8) a non-wild type level of an OCTlp-protein, 9) allelic loss of an OCTlp gene, and 10) inappropriate post-translational modification of an OCTlp-protein. As described herein, there are a large number of assay techniques known in the art, which can be used for detecting lesions in an OCTlp gene. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the OCTlp-gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to an OCTlp gene under conditions such that hybridization and amplification of the OCTlp-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an OCTlp gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in OCTlp can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in OCTlp can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the OCTlp gene and detect mutations by comparing the sequence of the sample OCTlp with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the OCTlp gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type OCTlp sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in OCTlp cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an OCTlp sequence, e.g., a wild-type OCTlp sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in OCTlp genes. For example, single strand conformation polymorphism (SSCP) may be used to detect differences in electrophoretic mobility between mutant and wild type nucleic acids (Orita et al. (1989)Proc Natl. Acad. Sci USA, 86:2766, see also Cotton (1993) Mutat. Res. 285:125-144; and Hayashi (1992) Genet Anal Tech Appl 9:73-79). Single-stranded DNA fragments of sample and control OCTlp nucleic acids will be denatured and allowed to renature. The secondary structure of single-stranded nucleic acids varies according to sequence, the resulting alteration in electrophoretic mobility enables the detection of even a single base change.

The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology, which depends on selective PCR amplification, may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments, amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an OCTlp gene.

Furthermore, any cell type or tissue in which OCTlp is expressed may be utilized in the prognostic assays described herein.

3. Pharmacogenomics

Agents, or modulators that have a stimulatory or inhibitory effect on OCTlp activity (e.g., OCTlp gene expression), as identified by a screening assay described herein, can be administered to individuals to treat (prophylactically or therapeutically) disorders (e.g., an immunological disorder) associated with aberrant OCTlp activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of OCT-like protein, the expression of OCTlp nucleic acid, or the content of mutations within OCTlp genes in an individual can be determined and used to select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated: genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes (CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers, who do not respond to standard doses. Recently, the molecular basis of ultra-rapid metabolism has been identified to be due to CYP2D6 gene amplification.

Thus, the activity of OCT-like protein, the expression of OCTlp nucleic acid, or the content of mutations within OCTlp genes in an individual can be determined and used to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an OCTlp modulator, such as a modulator identified by one of the exemplary screening assays described herein.

4. Monitoring of Effects During Clinical Trials

Monitoring the influence of agents (e.g., drugs, compounds) on the expression or activity of OCTlp (e.g., the ability to modulate aberrant cell proliferation and/or differentiation) can be applied not only in basic drug screening, but also in clinical trials. For example, the effectiveness of an agent determined by a screening assay as described herein to increase OCTlp gene expression, protein levels, or upregulate OCTlp activity, can be monitored in clinical trials of subjects exhibiting decreased OCTlp gene expression, protein levels, or downregulated OCTlp activity.

Alternatively, the effectiveness of an agent determined by a screening assay to decrease OCTlp gene expression, protein levels, or downregulated OCTlp activity, can be monitored in clinical trials of subjects exhibiting increased OCTlp gene expression, protein levels, or upregulated OCTlp activity. In such clinical trials, the expression or activity of OCTlp and, preferably, other genes that have been implicated in, for example, a cellular proliferation disorder can be used as a “read out” or markers of the immune responsiveness of a particular cell.

For example, and not by way of limitation, genes, including OCTlp, that are modulated in cells by treatment with an agent (e.g., a compound, drug or small molecule) that modulates OCTlp activity (e.g., identified in a screening assay as described herein) can be identified. Thus, to study the effect of agents on cellular proliferation disorders, for example, in a clinical trial, cells can be isolated and RNA prepared and analyzed for the levels of expression of OCTlp and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of OCTlp or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of an OCT-like protein, mRNA, or genomic DNA in the preadministration sample; (iii) obtaining one or more post-administration samples from the subject; (iv) detecting the level of expression or activity of the OCT-like protein, mRNA, or genomic DNA in the post-administration samples; (v) comparing the level of expression or activity of the OCT-like protein, mRNA, or genomic DNA in the pre-administration sample with the OCT-like protein, mRNA, or genomic DNA in the post administration sample or samples; and (vi) altering the administration of the agent to the subject accordingly. For example, increased administration of the agent may be desirable to increase the expression or activity of OCTlp to higher levels than detected, i.e., to increase the effectiveness of the agent. Alternatively, decreased administration of the agent may be desirable to decrease expression or activity of OCTlp to lower levels than detected, i.e., to decrease the effectiveness of the agent.

C. Methods of Treatment

The present invention provides for both prophylactic and therapeutic methods of treating a subject having or at risk of having (or susceptible to) a disorder associated with aberrant OCTlp expression or activity. Such disorders include chronic neurological disorders, neurodegenerative diseases (e.g., Alzheimer's Disease, Parkinson's Disease, Huntington's Disease, or amyotrophic lateral sclerosis), behavioral disorders, eating disorders, or sleep disorders and other disorders described herein.

1. Prophylactic Methods

In one aspect, the invention provides a method for preventing in a subject, a disease or condition associated with an aberrant OCTlp expression or activity, by administering to the subject an agent that modulates OCTlp expression or at least one OCTlp activity. Subjects at risk for a disease that is caused or contributed to by aberrant OCTlp expression or activity can be identified by, for example, any or a combination of diagnostic or prognostic assays as described herein. Administration of a prophylactic agent can occur prior to the manifestation of symptoms characteristic of the OCTlp aberrancy, such that a disease or disorder is prevented or, alternatively, delayed in its progression. Depending on the type of OCTlp aberrancy, for example, an OCTlp agonist or OCTlp antagonist agent can be used for treating the subject. The appropriate agent can be determined based on screening assays described herein.

2. Therapeutic Methods

Another aspect of the invention pertains to methods of modulating OCTlp expression or activity for therapeutic purposes. The modulatory method of the invention involves contacting a cell with an agent that modulates one or more of the activities of OCT-like protein activity associated with the cell. An agent that modulates OCT-like protein activity can be an agent as described herein, such as a nucleic acid or a protein, a naturally-occurring biological molecule that is transported by an OCT-like protein, a peptide, an OCTlp peptidomimetic, or other small molecule. In one embodiment, the agent stimulates one or more of the biological activities of OCT-like protein. Examples of such stimulatory agents include active OCT-like protein and a nucleic acid molecule encoding OCTlp that has been introduced into the cell. In another embodiment, the agent inhibits one or more of the biological activities of OCT-like protein. Examples of such inhibitory agents include antisense OCTlp nucleic acid molecules and anti-OCTlp antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of an OCT-like protein or nucleic acid molecule. In one embodiment, the method involves administering an agent (e.g., an agent identified by a screening assay described herein), or combination of agents that modulate (e.g., upregulate or downregulate) OCTlp expression or activity. In another embodiment, the method involves administering an OCT-like protein or nucleic acid molecule as therapy to compensate for reduced or aberrant OCTlp expression or activity.

Stimulation of OCTlp activity is desirable in situations in which OCTlp is abnormally downregulated and/or in which increased OCTlp activity is likely to have a beneficial effect. Conversely, inhibition of OCTlp activity is desirable in situations in which OCTlp is abnormally upregulated and/or in which decreased OCTlp activity is likely to have a beneficial effect.

This invention is further illustrated by the following examples, which should not be construed as limiting. The contents of all references, patents and published patent applications cited throughout this application are hereby incorporated by reference.

### EXAMPLES

### Example 1

### Isolation and Characterization of Human OCTlp cDNAs

A rat frontal cortex library was prepared and sequencing was performed on numerous clones. Partial sequence was identified that showed homology to the OCT family of proteins. The rat sequence was used to design primers, which were used to identify and isolate a human clone from a human fetal brain cDNA library. 5′ and 3′ RACE was performed using a ClonTech kit.

Complete sequencing of a human cDNA clone revealed an approximately 2.5 kb cDNA insert with a 1644 base pair open reading frame (excluding the triplet that constitutes the stop codon), which predicts a novel transmembrane protein.

### Example 2

### Distribution of OCTlp mRNA in Human Tissues

The expression of OCTlp was analyzed using Northern blot hybridization. A portion of OCTlp cDNA encoding the amino terminus of OCT-like protein was generated by PCR. The DNA was radioactively labeled with ³²P-dCTP using the Prime-It kit (Stratagene; La Jolla, Calif.) according to the instructions of the supplier. Filters containing human mRNA (MTNI and MTNII: Clontech; Palo Alto, Calif.) were probed in ExpressHyb hybridization solution (Clontech) and washed at high stringency according to manufacturer's recommendations.

These studies revealed that OCTlp is expressed as an approximately 3.0 kb OCTlp mRNA transcript in brain tissue. Under the same conditions of analysis, a lower level of expression of this transcript was observed in testes, while there appeared to be little or no expression in heart, spleen, lung, liver, skeletal muscle, or kidney.

### Example 3

### Characterization of OCT-like Proteins

The predicted amino acid sequence of human OCT-like protein was compared to amino acid sequences of known proteins and various motifs were identified. In addition, the molecular weight of the human OCT-like proteins was predicted.

The human OCTlp cDNA isolated as described above (FIG. 1; SEQ ID NO:1) encodes a 548 amino acid protein (FIG. 1; SEQ ID NO:2). The signal peptide prediction program SIGNALP Optimized Tool (Nielsen et al. (1997) Protein Engineering 10:1-6) predicted that OCTlp does not includes a signal peptide. OCTlp also included 12 predicted transmembrane domains (amino acids SEQ ID NOs. 6-17).

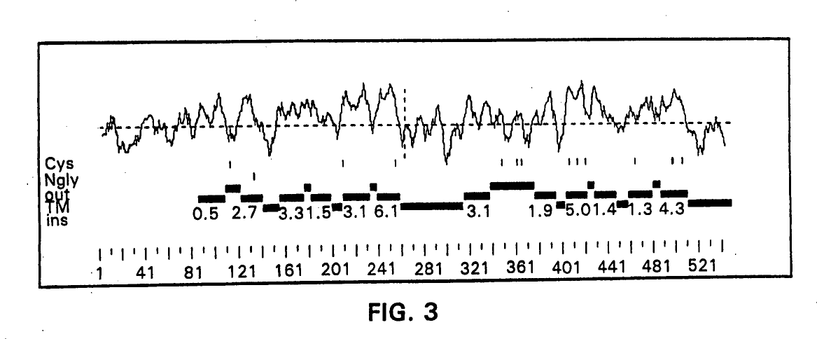

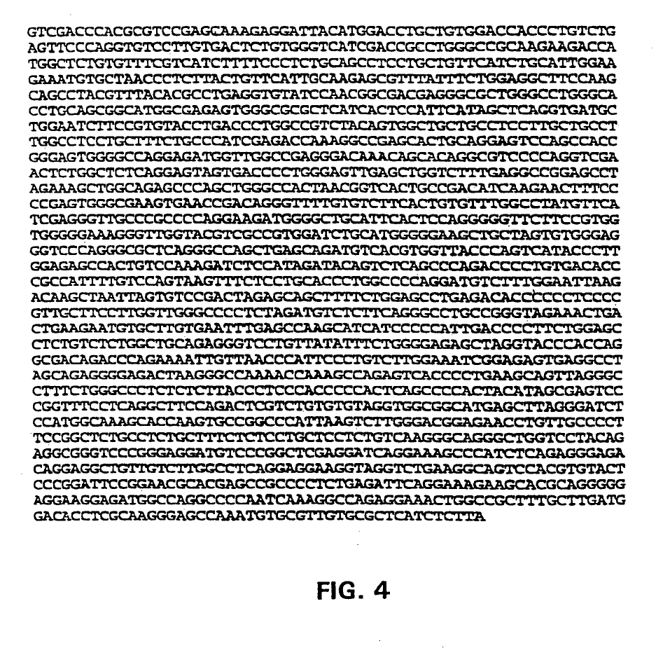

A hydropathy plot of OCTlp is presented in FIG. 3. This plot shows the twelve predicted TM domains as well as extracellular regions (labelled “OUT”; amino acids 109-121, 176-181, 233-238, 338-375, 422-427, and 480-486 of SEQ ID NO:2) and cytoplasmic regions (labelled “IN”; amino acids 1-85, 141-154, 199-208, 259-314, 395-402, 448-457, 511-548 of SEQ ID NO:2) as well as the location of cysteines (“cys”; short vertical lines just below plot). For general information regarding PFAM identifiers refer to Sonnhammer et al. (1997) Protein 28:405-420 and the internet site at http://www.psc.edu/general/software/packages/pfam/pfam.html.

As shown in FIG. 2, OCTlp has a region (amino acid residues 71 to 524 of SEQ ID NO:2; SEQ ID NO:4) of homology to a consensus sequence for sugar and other transporter molecules that was derived from a hidden Markov model (SEQ ID NO:5).

OCTlp has a predicted MW of 60 kDa.

### Example 4

### Preparation of OCT-like Proteins

Recombinant OCTlp can be produced in a variety of expression systems. For example, the mature OCTlp peptide can be expressed as a recombinant glutathione-S-transferase (GST) fusion protein in E. coli and the fusion protein can be isolated and characterized. Specifically, as described above, OCTlp can be fused to GST and this fusion protein can be expressed in E. coli strain PEB199. Expression of the GST-OCTlp fusion protein in PEB199 can be induced with IPTG. The recombinant fusion protein can be purified from crude bacterial lysates of the induced PEB199 strain by affinity chromatography on glutathione beads.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence that is at least 65% identical to the nucleotide sequence of SEQ ID NO:1 or SEQ ID NO:3, or a complement thereof;
b) a nucleic acid molecule comprising a fragment of at least 300 nucleotides of the nucleotide sequence of SEQ ID NO:1 or SEQ ID NO:3, or a complement thereof;
c) a nucleic acid molecule that encodes a polypeptide comprising the amino acid sequence of SEQ ID No:2;
d) a nucleic acid molecule that encodes a fragment of a polypeptide comprising the amino acid sequence of seq id no:2, wherein the fragment comprises at least 15 contiguous amino acid residues of SEQ ID NO:2; and
e) a nucleic acid molecule that encodes a naturally occurring allelic variant of a polypeptide comprising the amino acid sequence of SEQ ID NO:2, wherein the nucleic acid molecule hybridizes to a nucleic acid molecule comprising SEQ ID NO:1 or SEQ ID NO:3 under stringent conditions:

2. The isolated nucleic acid molecule of claim 1, which is selected from the group consisting of:
a) a nucleic acid molecule comprising the nucleotide sequence of SEQ ID NO:1 or SEQ ID NO:3 or a complement thereof; and
b) a nucleic acid molecule that encodes a polypeptide comprising the amino acid sequence of SEQ ID NO:2.

3. The nucleic acid molecule of claim 1, further comprising vector nucleic acid sequences.

4. The nucleic acid molecule of claim 1, further comprising nucleic acid sequences encoding a heterologous polypeptide.

5. A host cell containing the nucleic acid molecule of claim 1.

6. The host cell of claim 4, which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2, wherein the fragment comprises at least 15 contiguous amino acids of SEQ ID NO:2;
b) a naturally occurring allelic variant of a polypeptide comprising the amino acid sequence of SEQ ID NO:2, wherein the polypeptide is encoded by a nucleic acid molecule that hybridizes to a nucleic acid molecule comprising SEQ ID NO:1 or SEQ ID NO:3 under stringent conditions;
c) a polypeptide that is encoded by a nucleic acid molecule comprising a nucleotide sequence that is at least 60% identical to a nucleic acid molecule comprising the nucleotide sequence of SEQ ID NO:1 or SEQ ID NO:3.

9. The isolated polypeptide of claim 8, comprising the amino acid sequence of SEQ ID NO:2.

10. The polypeptide of claim 8, further comprising heterologous amino acid sequences.

11. An antibody that selectively binds to a polypeptide of claim 8.

12. A method for producing a polypeptide selected from the group consisting of:
a) a polypeptide comprising the amino acid sequence of SEQ ID NO:2;
b) a fragment of a polypeptide comprising the amino acid sequence of SEQ ID NO:2, wherein the fragment comprises at least 15 contiguous amino acids of SEQ ID NO:2; and
c) a naturally occurring allelic variant of a polypeptide comprising the amino acid sequence of SEQ ID NO:2, wherein the polypeptide is encoded by a nucleic acid molecule that hybridizes to a nucleic acid molecule comprising SEQ ID NO:1 or SEQ ID NO:3 under stringent conditions;
the method comprising culturing the host cell of claim 5 under conditions in which the nucleic acid molecule is expressed.

13. The isolated polypeptide of claim 8 comprising the amino acid sequence of SEQ ID NO:2.

14. A method for detecting the presence of a polypeptide of claim 8 in a sample, comprising:
a) contacting the sample with a compound that selectively binds to a polypeptide of claim 8; and
b) determining whether the compound binds to the polypeptide in the sample.

15. The method of claim 14, wherein the compound that binds to the polypeptide is an antibody.

16. A kit comprising a compound that selectively binds to a polypeptide of claim 8 and instructions for use.

17. A method for detecting the presence of a nucleic acid molecule of claim 1 in a sample, comprising the steps of:
a) contacting the sample with a nucleic acid probe or primer that selectively hybridizes to the nucleic acid molecule; and
b) determining whether the nucleic acid probe or primer binds to a nucleic acid molecule in the sample.

18. The method of claim 17, wherein the sample comprises mRNA molecules and is contacted with a nucleic acid probe.

19. A kit comprising a compound that selectively hybridizes to a nucleic acid molecule of claim 1 and instructions for use.

20. A method for identifying a compound that binds to a polypeptide of claim 8 comprising the steps of:
a) contacting a polypeptide of claim 8, or a cell expressing a polypeptide of claim 8, with a test compound; and
b) determining whether the polypeptide binds to the test compound.

21. The method of claim 20, wherein the binding of the test compound to the polypeptide is detected by a method selected from the group consisting of:
a) detection of binding by direct detecting of test compound/polypeptide binding; and
b) detection of binding using a competition binding assay.

22. A method for modulating the activity of a polypeptide of claim 8, the method comprising contacting a polypeptide of claim 8 or a cell expressing a polypeptide of claim 8 with a compound that binds to the polypeptide in a sufficient concentration to modulate the activity of the polypeptide.

23. A method for identifying a compound that modulates the activity of a polypeptide of claim 8, comprising:
a) contacting a polypeptide of claim 8 with a test compound; and
b) determining the effect of the test compound on the activity of the polypeptide to thereby identify a compound that modulates the activity of the polypeptide.


SEQUENCE LISTING (20 sequences)

SEQ ID NO: 1 - DNA, 2562 bp, Homo sapiens
  CDS (185)...(1828)
  misc_feature (1)...(2562): n = A,T,C or G
cccacgcgtccgggctttgtctcgtgggctggtccccagcggctccctccccgaacagctgctgctccagggaggaagcggcgcgggtgctgtccagcttcccggtgctgaaaaccggagggctcgtcatccaccactaccatgtaagggccatgagaagggctcatcctggcgcagcgcggacatggaggaggacttattccagctaaggcagctgccggttgtgaaattccgtcgcacaggcgagagtgcaaggtcagaggacgacacggcttcaggagagcatgaagtccagattgaaggggtccacgtgggcctagaggctgtggagctggatgatggggcagctgtgcccaaggagtttgccaatcccaccgatgatactttcatggtggaagatgcagtggaagccattggctttggaaaatttcagtggaagctgtctgttctcactggcttggcttggatggctgatgccatggagatgatgatcctcagcatcctggcaccacagctgcattgcgagtggaggctcccaagctggcaggtggcattgctgacctcggtggtctttgtaggcatgatgtccagctccacgctctggggaaatatctcagaccagtacggcaggaaaacagggctgaagatcagcgtgctgtggactctgtactatggcatccttagtgcatttgcgcccgtgtatagctggatcctggtgctccggggcctggtgggcttcgggatcggaggagttccccagtcggtgacgctgtatgccgagttccttcccatgaaagccagagctaaatgtattttgctgattgaggtattctgggccatcgggacagtgttcgaggtcgtcctggctgtgttcgtgatgcccagcctgggctggcgttggctgctcatcctctcagctgtcccgctcctcctctttgccgtgctgtgtttctggctgcctgaaagtgcaaggtatgatgtgctgtcagggaaccaggaaaaggcaatcgccaccttaaagaggatagcaactgaaaacggagctcccatgccgctggggaaactcatcatctccagacaggaagaccgaggcaaaatgagggaccttttcacaccccattttagatggacaactttgctgctgtggtttatatggttttccaatgcattctcttactacgggttagttctactcaccacagaactcttccaggcaggagatgtctgcggcatctccagtcggaagaaggctgtagaggcaaaatgcagcctggcctgcgagtacctgagtgaggaggattacatggacttgctgtggaccaccctctctgagtttccaggtgtccttgtgactctgtggattattgaccgcctggggcgcaagaagaccatggccctgtgctttgtcatcttctccttctgcagcctcctgctgtttatctgtgttggaagaaatgtgctcactctgttactcttcattgcaagagcgtttatttctggaggctttcaagcggcatatgtttacacacctgaggtctaccccacggcaacgcgggccctcggcctgggcacctgcagcggcatggcaagagtgggtgctctcatcactccgttcatcgcccaggtgatgctggaatcctctgtgtacctgactctggcagtttacagtggctgctgcctcctggctgccctggcctcctgctttttgcccattgagaccaaaggcggaggactgcaggagtccagccaccgggagtggggccaggagatggtcggccgaggaatgcacggtgcaggtgttaccaggtcgaactctggctctcaggaatagtgaccgatgggggactgagctggtctttgaggctgcagagcttggggggctggcaggccccaactggggcactgattgtcactgccgacatcaagaactcacccaagagtatgacctggaccaacagggttttgtgtcttgactcagtttgctcatcttcattgaggtccacccagggatggggagatgtttgctctagggggttctctgtatatgtggtgaaagctttgttcataacctgtggatctacatgggaagactacccatattaggagggtctggtaatgccagcaaccaatcagacaccacccagagtcacccggccaaaccctcagtgaacaaccaaaatatctctctgtagataccgtccaggctcaggcccatgtgacacctgctgtccacccaccggacctgttcagtaggtttctcccacacccacagccccaggctttcttctttgaaattgcaggcgatctaggtgtggtctgagcagctatttcctggcaggggccccccggtttgcctccctagagcctgaccagtggattctctggcagatggacatggtgcattcaaactggagccacatgcccccacccagcccctnttggagttgcccgttgttggcaccaagagatccagatgtgtcctggggacagctgggtcttgcaccaggtgacaacctcaaaacgccgttaccccctggggaactgaggactgagngccaagtg

SEQ ID NO: 2 - protein, 548 aa, Homo sapiens
MEEDLFQLRQLPVVKFRRTGESARSEDDTASGEHEVQIEGVHVGLEAVELDDGAAVPKEFANPTDDTFMVEDAVEAIGFGKFQWKLSVLTGLAWMADAMEMMILSILAPQLHCEWRLPSWQVALLTSVVFVGMMSSSTLWGNISDQYGRKTGLKISVLWTLYYGILSAFAPVYSWILVLRGLVGFGIGGVPQSVTLYAEFLPMKARAKCILLIEVFWAIGTVFEVVLAVFVMPSLGWRWLLILSAVPLLLFAVLCFWLPESARYDVLSGNQEKAIATLKRIATENGAPMPLGKLIISRQEDRGKMRDLFTPHFRWTTLLLWFIWFSNAFSYYGLVLLTTELFQAGDVCGISSRKKAVEAKCSLACEYLSEEDYMDLLWTTLSEFPGVLVTLWIIDRLGRKKTMALCFVIFSFCSLLLFICVGRNVLTLLLFIARAFISGGFQAAYVYTPEVYPTATRALGLGTCSGMARVGALITPFIAQVMLESSVYLTLAVYSGCCLLAALASCFLPIETKGGGLQESSHREWGQEMVGRGMHGAGVTRSNSGSQE

SEQ ID NO: 3 - DNA, 1644 bp, Homo sapiens
atggaggaggacttattccagctaaggcagctgccggttgtgaaattccgtcgcacaggcgagagtgcaaggtcagaggacgacacggcttcaggagagcatgaagtccagattgaaggggtccacgtgggcctagaggctgtggagctggatgatggggcagctgtgcccaaggagtttgccaatcccaccgatgatactttcatggtggaagatgcagtggaagccattggctttggaaaatttcagtggaagctgtctgttctcactggcttggcttggatggctgatgccatggagatgatgatcctcagcatcctggcaccacagctgcattgcgagtggaggctcccaagctggcaggtggcattgctgacctcggtggtctttgtaggcatgatgtccagctccacgctctggggaaatatctcagaccagtacggcaggaaaacagggctgaagatcagcgtgctgtggactctgtactatggcatccttagtgcatttgcgcccgtgtatagctggatcctggtgctccggggcctggtgggcttcgggatcggaggagttccccagtcggtgacgctgtatgccgagttccttcccatgaaagccagagctaaatgtattttgctgattgaggtattctgggccatcgggacagtgttcgaggtcgtcctggctgtgttcgtgatgcccagcctgggctggcgttggctgctcatcctctcagctgtcccgctcctcctctttgccgtgctgtgtttctggctgcctgaaagtgcaaggtatgatgtgctgtcagggaaccaggaaaaggcaatcgccaccttaaagaggatagcaactgaaaacggagctcccatgccgctggggaaactcatcatctccagacaggaagaccgaggcaaaatgagggaccttttcacaccccattttagatggacaactttgctgctgtggtttatatggttttccaatgcattctcttactacgggttagttctactcaccacagaactcttccaggcaggagatgtctgcggcatctccagtcggaagaaggctgtagaggcaaaatgcagcctggcctgcgagtacctgagtgaggaggattacatggacttgctgtggaccaccctctctgagtttccaggtgtccttgtgactctgtggattattgaccgcctggggcgcaagaagaccatggccctgtgctttgtcatcttctccttctgcagcctcctgctgtttatctgtgttggaagaaatgtgctcactctgttactcttcattgcaagagcgtttatttctggaggctttcaagcggcatatgtttacacacctgaggtctaccccacggcaacgcgggccctcggcctgggcacctgcagcggcatggcaagagtgggtgctctcatcactccgttcatcgcccaggtgatgctggaatcctctgtgtacctgactctggcagtttacagtggctgctgcctcctggctgccctggcctcctgctttttgcccattgagaccaaaggcggaggactgcaggagtccagccaccgggagtggggccaggagatggtcggccgaggaatgcacggtgcaggtgttaccaggtcgaactctggctctcaggaa

SEQ ID NO: 4 - protein, 273 aa, Homo sapiens
EDAVEAIGFGKFQWKLSVLTGLAWMADAMEMMILSILAPQLHCEWRLPSWQVALLTSVVFVGMMSSSTLWGNISDQYGRKTGLKISVLWTLYYGILSAFAPVYSWILVLRGLVGFGIGGVPQSVTLYAEFLPMKARAKCILLIEVFWAIGTVFEVVLAVFVMPSLGWRWLLILSAVPLLLFAVLCFWLPESARYDVLSGNQEKAIATLKRIATENGAPMPLGKLIISRQEDRGKMRDLFTPHFRWTTLLLWFIWFSNAFSYYGLVLLTTELFQ

SEQ ID NO: 5 - protein, 293 aa, Homo sapiens
WCFMAAFGGMFMFGWDTGVINGFVWMIDFHYRFGMMHYDWTYYLSTMRWGLIVSIFNIGCMIGSIFFGWIGDMYGRRMSMMMVNVIFIIGIIIMIFSINYSWWMYIIGRIIMGIGVGGISVLVPMYISEIAPKHLRGTMVSWYQLMITIGIFIAYCFNYGFNYYNNDSWQWRWPLGLCFIWAIFMIIGMMFLPESPRWLVIKGRIEEARRSLQRLRGWDDVDPEIQEMMDEIEAMIEEELAGNASWGELFRRRTPKMRWRIIMGMMIQIFQQFTGINYIMYYSTTIFESVGMQ

SEQ ID NO: 6 - protein, 23 aa, Homo sapiens
LSVLTGLAWMADAMEMMILSILA

SEQ ID NO: 7 - protein, 19 aa, Homo sapiens
VALLTSVVFVGMMSSSTLW

SEQ ID NO: 8 - protein, 21 aa, Homo sapiens
ISVLWTLYYGILSAFAPVYSW

SEQ ID NO: 9 - protein, 17 aa, Homo sapiens
LVGFGIGGVPQSVTLYA

SEQ ID NO: 10 - protein, 24 aa, Homo sapiens
CILLIEVFWAIGTVFEVVLAVFVM

SEQ ID NO: 11 - protein, 20 aa, Homo sapiens
WLLILSAVPLLLFAVLCFWL

SEQ ID NO: 12 - protein, 23 aa, Homo sapiens
WTTLLLWFIWFSNAFSYYGLVLL

SEQ ID NO: 13 - protein, 19 aa, Homo sapiens
LLWTTLSEFPGVLVTLWII

SEQ ID NO: 14 - protein, 19 aa, Homo sapiens
MALCFVIFSFCSLLLFICV

SEQ ID NO: 15 - protein, 20 aa, Homo sapiens
LLLFIARAFISGGFQAAYVY

SEQ ID NO: 16 - protein, 22 aa, Homo sapiens
ALGLGTCSGMARVGALITPFIA

SEQ ID NO: 17 - protein, 24 aa, Homo sapiens
VYLTLAVYSGCCLLAALASCFLPI

SEQ ID NO: 18 - DNA, 24 bp, Homo sapiens
tccatgtccgcgctgcgccaggat

SEQ ID NO: 19 - DNA, 19 bp, Homo sapiens
ttagctggaataagtcctc

SEQ ID NO: 20 - DNA, 1906 bp, Rattus rattus
gtcgacccacgcgtccgagcaaagaggattacatggacctgctgtggaccaccctgtctgagttcccaggtgtccttgtgactctgtgggtcatcgaccgcctgggccgcaagaagaccatggctctgtgtttcgtcatcttttccctctgcagcctcctgctgttcatctgcattggaagaaatgtgctaaccctcttactgttcattgcaagagcgtttatttctggaggcttccaagcagcctacgtttacacgcctgaggtgtatccaacggcgacgagggcgctgggcctgggcacctgcagcggcatggcgagagtgggcgcgctcatcactccattcatagctcaggtgatgctggaatcttccgtgtacctgaccctggccgtctacagtggctgctgcctccttgctgccttggcctcctgctttctgcccatcgagaccaaaggccgagcactgcaggagtccagccaccgggagtggggccaggagatggttggccgagggacaaacagcacaggcgtccccaggtcgaactctggctctcaggagtagtgacccctgggagttgagctggtctttgaggccggagcctagaaagctggcagagcccagctgggccactaacggtcactgccgacatcaagaactttccccgagtgggcgaagtgaaccgacagggttttgtgtcttcactgtgtttggcctatgttcatcgagggttgcccgccccaggaagatggggctgcattcactccagggggttcttccgtggtgggggaaagggttggtacgtcgccgtggatctgcatgggggaagctgctagtgtgggagggtcccagggcgctcagggccagctgagcagatgtcacgtggttacccagtcatacccttggagagccactgtccaaagatctccatagatacagtctcagcccagacccctgtgacacccgccattttgtccagtaagtttctcctgcaccctggccccaggatgtctttggaattaagacaagctaattagtgtccgactagagcagcttttctggagcctgagacacccccctccccgttgcttccttggttgggcccctctagatgtctcttcagggcctgccgggtagaaactgactgaagaatgtgcttgtgaatttgagccaagcatcatcccccattgaccccttctggagcctctgtctctggctgcagagggtcctgttatatttctggggagagctaggtacccaccaggcgacagacccagaaaattgttaacccattccctgtcttggaaatcggagagtgaggcctagcagaggggagactaagggccaaaaccaaagccagagtcacccctgaagcagttagggcctttctgggccctctctcttaccctcccacccccactcagccccactacatagcgagtcccggtttcctcaggcttccagactcgtctgtgtgtaggtggcggcatgagcttagggatctccatggcaaagcaccaagtgccggcccattaagtcttgggacggagaacctgttgccccttccggctctgcctctgctttctctcctgctcctctgtcaagggcagggctggtcctacagaggcgggtcccgggaggatgtcccggctcgaggatcaggaaagcccatctcagagggagacaggaggctgttgtcttggcctcaggaggaaggtaggtctgaaggcagtccacgtgtactcccggattccggaacgcacgagccgcccctctgagattcaggaaagaagcacgcagggggaggaaggagatggccaggccccaatcaaaggccagaggaaactggccgctttgcttgatggacacctcgcaagggagccaaatgtgcgttgtgcgctcatctctta